(19) United States
(12) Patent Application Publication
(10) Pub. No.: US 2022/0033816 A1
(43) Pub. Date: Feb. 3, 2022

(54) USE OF miRNA 148 CLUSTER AS MARKER FOR DIAGNOSING AND/OR TREATING COGNITIVE IMPAIRMENT-ASSOCIATED DISEASES

(71) Applicant: Institute of Medicinal Biotechnology, Chinese Academy of Medical Sciences, Beijing (CN)

(72) Inventors: Rui LIU, Beijing (CN); Zhuorong LI, Beijing (CN); Hailun JIANG, Beijing (CN); Li ZENG, Beijing (CN); Linlin WANG, Beijing (CN)

(21) Appl. No.: 17/347,978

(22) Filed: Jun. 15, 2021

(30) Foreign Application Priority Data
Jul. 28, 2020 (CN) 202010738200.2

(51) Int. Cl. C12N 15/113; C12Q 1/6883

(52) U.S. Cl. CPC C12N 15/113; C12Q 1/6883; C12N 2310/141; C12Q 2600/158; C12N 2320/51; C12Q 2600/178; C12N 2320/30

(57) ABSTRACT

The present disclosure discloses use of a miRNA 148 cluster as a marker for diagnosing and/or treating cognitive impairment-associated diseases. The present disclosure provides use of the miRNA 148 cluster in the diagnosis and treatment of cognitive impairment-associated diseases. The expression level of the miRNA 148 cluster is detected using primers for the microRNA through cognitive impairment-associated disease models, and it is found that the expression of the miRNA 148 cluster is significantly reduced during the progression of the cognitive impairment-associated diseases. The miRNA 148 cluster is found to directly target p35 to inhibit hyperphosphorylation of Tau and can be negatively regulated by the upregulated PTEN in AD pathology. Upregulation of miRNA 148 cluster improves the cognitive dysfunction and inhibit the hyperphosphorylation of Tau in AD pathology, in which the PTEN/Akt/CREB and p35/CDK signaling pathways play a key role.

### CROSS-REFERENCE TO RELATED APPLICATIONS

This application claims priority to Chinese Application No. 202010738200.2, filed Jul. 28, 2020, the contents of which are incorporated by reference herein in its entirety.

### TECHNICAL FIELD

The present disclosure relates to the field of biotechnology, and in particular to use of a miRNA 148 cluster as a marker for diagnosing and/or treating cognitive impairment-associated diseases.

### BACKGROUND

Neurocognitive disorder (NCD) is a group of syndromes with cognitive deficits as main clinical manifestations, including disorders of thought, reasoning, memory and problem solving. According to “Diagnostic and Statistical Manual of Mental Disorders, Fifth Edition (DSM-5)”, cognitive impairment is divided into mild cognitive impairment (MCI) and severe cognitive impairment (dementia). Cognitive impairment involves many brain and physical diseases, where Alzheimer's disease (AD) and vascular dementia (VaD) are the most common cognitive impairment-associated diseases. Cognitive impairment, which is more likely to affect the elderly, is not a part of a normal aging process, but a disease that occurs after the brain undergoes underlying pathological damage. Therefore, cognitive impairment also affects young people.

The prevalence of AD accounts for more than 50% of dementia, and the principal pathological features of AD are senile plaques formed due to extracellular amyloid deposition and neurofibrillary tangles formed due to intracellular Tau hyperphosphorylation. The incidence of VaD is second only to AD, accounting for about 15% to 20% of dementia. VaD is caused by ischemic stroke, hemorrhagic stroke, cerebral ischemia and hypoxia, or the like. The pathogenesis of these two diseases is relatively complex, and there is a lack of effective medicine and simple and non-invasive early diagnosis and screening methods. Therefore, seeking reliable diagnostic markers and effective drugs is a scientific problem to be solved urgently in the prevention and treatment of AD and VaD at present. However, there are no related gene reports on sporadic AD and VaD, which brings great difficulty to disease screening and prevention. Therefore, studying changes of related genes in the diseases is of great significance for the prevention and treatment of cognitive impairment-associated diseases and the discovery of clinical biomarkers.

### SUMMARY

In view of this, the present disclosure provides use of a miRNA 148 cluster as a marker for diagnosing and/or treating cognitive impairment-associated diseases.

To achieve the above objective, the present disclosure provides the following technical solutions.

The present disclosure provides use of a miRNA 148 cluster or an expression promoter thereof in the following (a), (b), (c), and/or (d):

(a) preparation of a substance that can inhibit phosphorylation of Tau;

(b) preparation of a substance that can alleviate neurodegeneration and has a neuroprotective effect;

(c) preparation of a substance for diagnosing and/or treating cognitive impairment-associated diseases; and

(d) preparation of a substance for reducing the expression of p35, p25, and cyclin-dependent kinase 5 (CDK5).

In an example of the present disclosure, the miRNA148 cluster is selected from hsa-miR-148a, with a nucleotide sequence shown in SEQ ID NO. 1; and the miRNA148 cluster is selected from hsa-miR-148a-3p, with a nucleotide sequence shown in SEQ ID NO. 2.

The present disclosure further provides a product with an active ingredient of a miRNA 148 cluster or an expression promoter thereof, and use of the product includes the following (a), (b), (c) and/or (d):

(a) inhibiting phosphorylation of Tau;

(b) alleviating neurodegeneration and providing a neuroprotective effect;

(c) diagnosing and/or treating cognitive impairment-associated diseases; and

(d) reducing the expression of p35, p25, and CDK5.

In an example of the present disclosure, the product for diagnosing cognitive impairment-associated diseases is a detection kit;

the detection kit includes primers of the miRNA 148 cluster; and the kit is used to diagnose cognitive impairment-associated diseases, predict the risk of developing cognitive impairment-associated diseases, or predict the outcome of cognitive impairment-associated diseases in patients suffering from or at risk of developing cognitive impairment-associated diseases.

In an example of the present disclosure, the primer is used to determine an expression level of the miRNA 148 cluster in a sample.

In an example of the present disclosure, the expression level of the miRNA 148 cluster is based on an expression level of the miRNA 148 cluster in a patient and a reference expression level of the miRNA 148 cluster in a healthy subject; and

if the expression level of the miRNA 148 cluster is significantly lower than the reference expression level of the miRNA 148 cluster in a healthy subject, it indicates that the patient has or is at risk of developing a cognitive impairment-associated disease.

In an example of the present disclosure, the expression level of the miRNA 148 cluster is determined by a sequencing-based method, an array-based method, or a PCR-based method.

In an example of the present disclosure, the expression promoter of the miRNA 148 cluster is at least a reagent, a medicament, a preparation, and a gene sequence that promote the expression or activation of Akt, a reagent, a medicament, a preparation, and a gene sequence that promote the expression or activation of cAMP-response element binding protein (CREB), and a reagent, a medicament, a preparation, and a gene sequence that inhibit the expression or activation of PTEN;

the Akt and CREB up-regulate the expression of the miRNA 148 cluster; and the PTEN downregulates the expression of the miRNA 148 cluster.

Use of an agonist for a miRNA 148 cluster in the preparation of a medicament for treating cognitive impairment-associated diseases also belongs to the protection scope of the present disclosure.

Use of a long non-coding RNA (lncRNA) interactive with a miRNA 148 cluster in the preparation of a medicament for treating cognitive impairment-associated diseases also belongs to the protection scope of the present disclosure.

In the present disclosure, the expression of a miRNA of the miRNA 148 cluster is reduced in AD and VaD, and miRNA 148 cluster reduces the phosphorylation level of Tau by targeting p35 in AD to play a role in improving cognitive dysfunction. The miRNA of the miRNA 148 cluster is:

(1) The miRNA 148 cluster is selected from the following: (a) classification of microRNA, where, the miRNA 148a is selected from hsa-miR-148a, with a sequence shown in SEQ ID NO. 1: gaggcaaagu ucugagacac uccgacucug aguaugauag aagucagugc acuacagaac uuugucuc, and a default mature body (hsa-miR-148a-3p) thereof has a sequence shown in SEQ ID NO. 2: ucagugcacuacagaacuuugu; and (b) modified derivatives of microRNAs; or microRNAs or modified miRNA derivatives with the same or substantially the same functions as microRNAs length of 18 nt to 26 nt.

The present disclosure further provides a preparation and a medicament, which are agonists for the microRNA in (1).

The present disclosure further provides a lncRNA, which is a lncRNA that specifically interacts with the microRNA in (1).

The present disclosure has the following advantages:

The present disclosure finds that the miRNA 148 cluster plays a role in the diagnosis and treatment of cognitive impairment-associated diseases. The expression level of the miRNA 148 cluster is detected using primers and/or probes for the microRNA through cognitive impairment-associated disease models, and it is found that the expression of the miRNA 148 cluster is significantly reduced during the progression of the cognitive impairment-associated disease. Therefore, the miRNA 148 cluster can be used as a novel marker for the auxiliary diagnosis of cognitive impairment-associated diseases.

The present disclosure finds that the miRNA 148 cluster participates in the pathological processes of AD and VaD, and exhibits a neuroprotective effect in AD and VaD cell models. In the pathological process of AD, the miRNA 148 cluster can directly bind to the 3′UTR of p35 mRNA to regulate the translation of p35, thereby secondarily regulating the expression of p25 and CDK5, reducing the phosphorylation level of Tau, and improving cognitive impairment in mice. CREB can directly bind to the promoter of miR-148a and upregulate the transcription of miR-148a. The PTEN/Akt signaling pathway can regulate the expression of miR-148a by regulating CREB, thereby affecting the phosphorylation level of Tau and improving the learning and memory capabilities of mice.

The present disclosure investigates functions of the miRNA 148 cluster deeply and systematically. Based on the above findings, the miRNA 148 cluster can be used as a novel therapeutic target for cognitive impairment-associated diseases, providing a new idea for targeted therapy using the miRNA 148 cluster as a biomarker for cognitive impairment-associated diseases.

### BRIEF DESCRIPTION OF THE DRAWINGS

In order to more clearly illustrate the implementations of the present disclosure or the technical solutions in the prior art, the following will briefly introduce the drawings that need to be used in the description of the implementations or the prior art. Obviously, the drawings in the following description are only exemplary. For those of ordinary skill in the art, other implementation drawings can be derived from the provided drawings without creative work.

The structure, scale, size, and the like shown in the drawings of this specification are only used to match the content disclosed in the specification and for those skilled in the art to understand and read, which are not used to limit the limitations for implementing the present disclosure and thus are not technically substantial. Any structural modification, scaling relation change, or size adjustment made without affecting the effects and objectives that can be achieved by the present disclosure shall fall within the scope that can be encompassed by the technical content disclosed in the present disclosure.

In order to more clearly illustrate the implementations of the present disclosure or the technical solutions in the prior art, the following will briefly introduce the drawings that need to be used in the description of the implementations or the prior art. Obviously, the drawings in the following description are only exemplary. For those of ordinary skill in the art, other implementation drawings can be derived from the provided drawings without creative work.

FIG. 1 shows the decreased expression of miR-148a of the present disclosure in brain tissues of APP/PS1 double-transgenic animals and wild-type (WT) animals detected by the miRNA microarray technology;

FIG. 2, panels A-H shows the decreased expression of the miR-148a of the present disclosure in cognitive impairment-associated disease models;

FIG. 3, panels A-E shows the protective effect of the miR-148a of the present disclosure on nerve cells and the inhibitory effect of Tau phosphorylation;

FIG. 4, panels A-N shows that the miR-148a of the present disclosure specifically binds to 3′UTR of p35 mRNA and downregulates the expression of p35, thereby regulating CDK5 and Tau protein phosphorylation;

FIG. 5, panels A-G shows that the miR-148a of the present disclosure improves the cognitive and memory dysfunction of AD mice, which relies on p35 to significantly reduce the Tau phosphorylation level in the brain of AD mice;

FIG. 6, panels A-H shows the increased expression of PTEN in the brains of AD model animals and senescence-accelerated mouse prone 8 (SAMP8) detected by the miRNA microarray technology and the Tau hyperphosphorylation caused by the up-regulation of PTEN expression in AD model cells;

FIG. 7, panels A-H shows that the expression of the miR-148a of the present disclosure is regulated by the PTEN/Akt signaling pathway;

FIG. 8, panels A-M shows that CREB specifically binds to the promoter of miR-148a of the present disclosure and regulates the transcription thereof, and the expression of CREB is regulated by the PTEN/Akt signaling pathway; and

FIG. 9, panels A-I shows that the expression of PTEN in the brain of AD mice can affect the learning and memory capacity of mice, and the inhibition of PTEN expression in the brain of AD mice can increase the expression of miR-148a, activate the Akt/CREB signaling pathway, and reduce the Tau phosphorylation level.

### DETAILED DESCRIPTION

The implementation of the present disclosure will be illustrated below in conjunction with specific examples. Those skilled in the art can easily understand other advantages and effects of the present disclosure from the content disclosed in this specification. Obviously, the described examples are merely a part rather than all of the examples of the present disclosure. All other examples obtained by a person of ordinary skill in the art based on the examples of the present disclosure without creative efforts shall fall within the protection scope of the present disclosure.

In the present disclosure, the term “expression level” refers to a measured expression level compared with a reference nucleic acid (for example, from a control), or a calculated average expression value (for example, in RNA microarray analysis). A specified “expression level” can also be used as a result and determined by the comparison and measurement of a plurality of nucleic acids of interest disclosed below, and show the relative abundance of these transcripts with each other. The expression level can also be evaluated relative to the expression of different tissues, patients versus healthy controls, etc.

In the context of the present disclosure, a “sample” or “biological sample” is a sample that is derived from or has been in contact with a biological organism. Examples of biological samples include: cells, tissues, body fluids, biopsy samples, blood, urine, saliva, sputum, plasma, serum, cell culture supernatant, etc.

A “gene” is a nucleic acid segment that carries the information necessary to produce a functional RNA product in a controlled manner. A “gene product” is a biomolecule produced by gene transcription or expression, such as mRNA or translated protein.

“miRNA” is a short, naturally occurring RNA molecule, and should have the general meaning understood by those skilled in the art. A “miRNA-derived molecule” is a molecule obtained from a miRNA template chemically or enzymatically, such as cDNA.

“lncRNA” is a non-coding or slightly-coding RNA molecule with a length of more than 200 bases, and should have the general meaning understood by those skilled in the art. lncRNA can interact with miRNA as a competitive endogenous RNA (ceRNA), participate in the regulation of target genes, and play an important role in the occurrence and development of diseases.

In the present disclosure, the term “array” refers to an arrangement of addressable positions on a device (such as a chip device). The number of locations can vary from a few to at least hundreds or thousands. Each position represents an independent reaction site. Arrays include, but are not limited to, nucleic acid arrays, protein arrays, and antibody arrays. “Nucleic acid array” refers to an array including nucleic acid probes, such as oligonucleotides, polynucleotides, or large portions of genes. The nucleic acids on the array are preferably single-stranded.

“PCR-based method” refers to a method involving polymerase chain reaction (PCR). This is a method of exponentially amplifying nucleic acids “such as DNA or RNA” by using one, two or more primers to replicate enzymatically in vitro. For RNA amplification, reverse transcription can be used as the first step. PCR-based methods include kinetic or quantitative PCR (qPCR), which are particularly suitable for analyzing expression levels. When it achieves the determination of the expression level, for example, a PCR-based method can be used to detect the presence of a given mRNA, which reverse transcribes a complete mRNA library (the so-called transcriptome) into cDNA with the help of reverse transcriptase, and the presence of a given cDNA is detected with the help of corresponding primers. This method is commonly referred to as reverse transcriptase PCR (RT-PCR).

In the present disclosure, the term “PCR-based method” includes both end-point PCR applications and kinetic/real-time PCR techniques using special fluorophores or intercalating dyes, which emit fluorescent signals as functions of amplification targets and allow monitoring and quantification of the targets.

In the present disclosure, the term “marker” or “biomarker” refers to a biomolecule whose presence or concentration can be detected and associated with a known condition (such as a disease state) or clinical outcome (such as response to treatment), such as nucleic acids, peptides, proteins, and hormones.

In the present disclosure, miRNA has the advantages of being endogenous, small in size and easy to pass through the blood-brain barrier (BBB), which can not only regulate translation and expression by binding to target genes, but also interact with lncRNA. Moreover, a single miRNA may interact with multiple target genes and lncRNAs, and multiple miRNAs may also interact with the same target gene or lncRNA to form a complex regulatory network in the brain.

### Example 1 Detection of Aberrant Expression of miR-148a in Brain Tissues of AD Model Animals and WT Control Animals by miRNA Microarray Technology

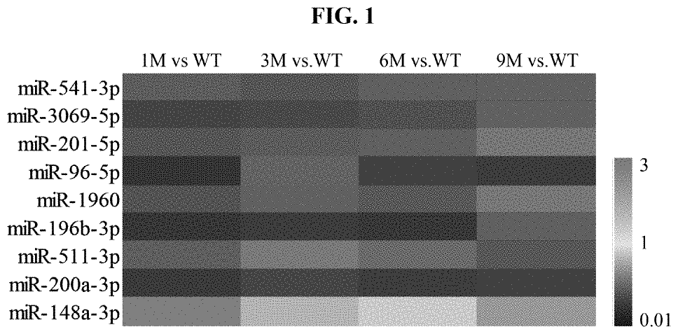

RNA was extracted from the brain tissues of 1-month-old, 3-month-old, 6-month-old, and 9-month-old APP/PS1 double-transgenic mice and WT control mice, and the miRNA was fluorescently labeled with the miRCURY™ Array Power Labeling kit. The miRNA 148 cluster of the present disclosure was hsa-miR-148a, with a sequence shown in SEQ ID NO. 1: gaggcaaagu ucugagacac uccgacucug aguaugauag aagucagugc acuacagaac uuugucuc. A default mature body (hsa-miR-148a-3p) thereof had a sequence shown in SEQ ID NO. 2: ucagugcacuacagaacuuugu. The mature body (hsa-miR-148a-3p) miRNA involved: reverse transcription primer: SEQ ID NO. 3: gtcgtatcca gtgcagggtc cgaggtattc gcactggata cgacacaaag; qPCR forward primer: SEQ ID NO. 4: gcgcgtcagt gcactacagaa; and reverse primer: SEQ ID NO. 5: agtgcagggt ccgaggtatt. Then the sample was hybridized on the miRCURY™ Array. A microarry was scanned with Axon GenePix 4000B microarray scanner, and the original data were analyzed with GenePix pro V6.0 software. As shown in the array results in FIG. 1, 9 miRNAs continuously changed in the brains of the 1-month-old, 3-month-old, 6-month-old, and 9-month-old APP/PS1 mice, which may be involved in the entire pathology of AD. The expression of miR-148a in the brains of the 1-month-old, 3-month-old, 6-month-old, and 9-month-old APP/PS1 mice was continuously downregulated, and the difference was the most significantly (mean±SEM, n=3, fold change>2), suggesting that miR-148a is closely related to AD.

### Example 2 Aberrant Expression of miR-148a in the In Vitro AD Model

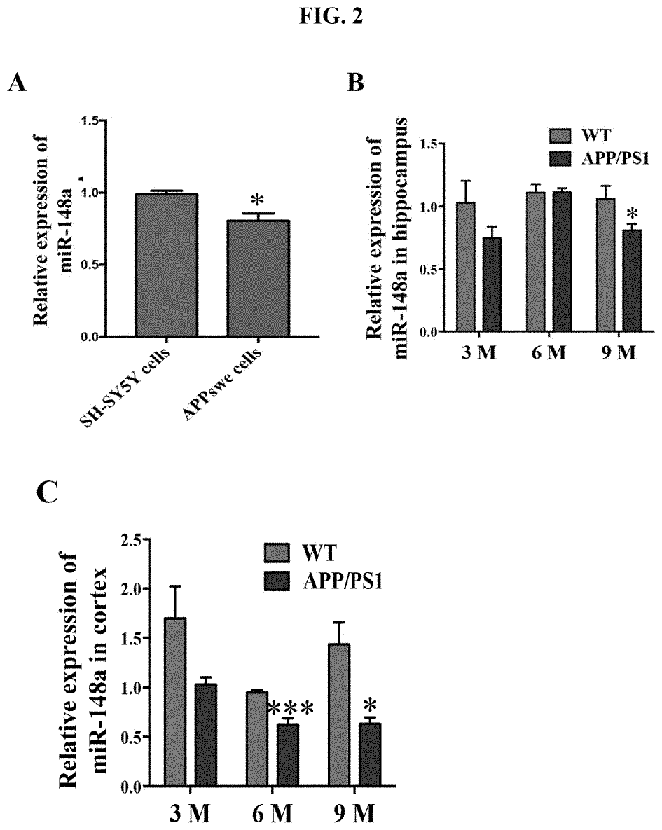

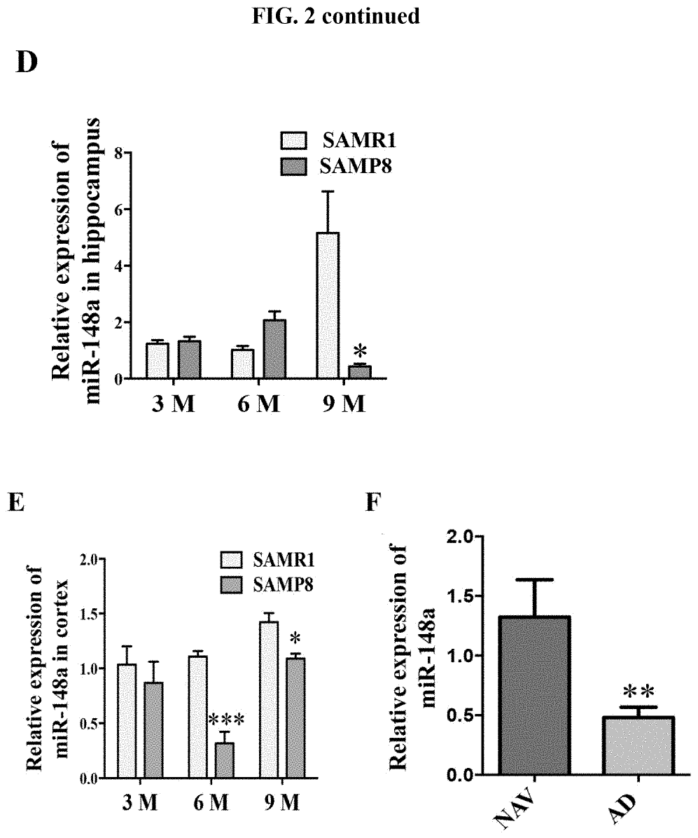

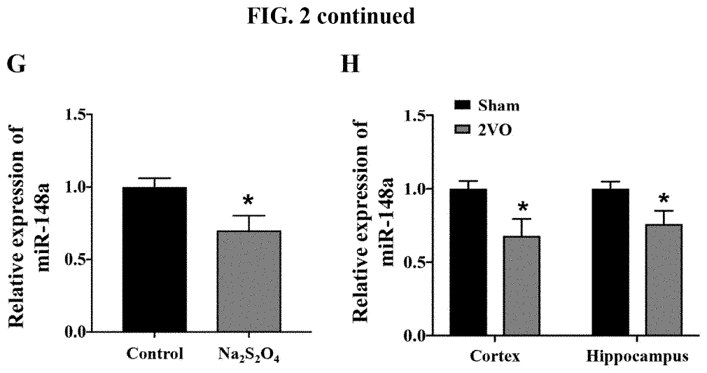

The Swedish mutant APP gene was stably transfected into SH-SYSY cells to construct a stable transgenic APPswe cell line. The present disclosure used 300 μM Cu2+ (CuSO4) to damage APPswe cells to establish an AD cell model, and cellular RNA was extracted to detect the expression level of miR-148a. As shown in FIG. 2, panel A, compared with normal control SH-SY5Y cells, the expression of miR-148a was significantly downregulated in AD model cells (mean±SEM, n=3, *P<0.05).

### Example 3 Aberrant Expression of miR-148a in AD Model Animals and Senescence-Accelerated Prone Mice SAMP8 Strain

qPCR was used to detect the level of miR-148a expression in the brains of APP/PS1 double-transgenic mice and WT control mice thereof, and SAMP8 mice and control mice thereof (SAMR1). As shown in FIG. 2, panels B-C, in the cortex and hippocampus of 3-month-old, 6-month-old, and 9-month-old APP/PS1 mice, the expression of miR-148a showed a downward trend, where, 6-month-old and 9-month-old APP/PS1 mice exhibited statistical differences from WT mice at the same age (mean±SEM, n=4, *P<0.05, ***P<0.001). As shown in FIG. 2, panels D-E, in the hippocampus of SAMP8 mice, the expression of miR-148a in the hippocampus of 9-month-old mice was significantly decreased than that of the SAMR1 control mice at the same age (mean±SEM, n=4, *P<0.05); and in the cortex, the expression of miR-148a in 6-month-old and 9-month-old SAMP8 mice was significantly decreased than that of SAMR1 control mice (mean±SEM, n=4, *P<0.05, ***P<0.001). It can be concluded that miR-148a is downregulated during AD pathology or aging.

### Example 4 Changes of miR-148a Level in the Serum of AD Patients

To confirm the correlation between the miR-148a level and AD, miRNA was extracted from the serum of fourteen AD patients and five health age-matched volunteers (HAVs), and the expression of miR-148a was detected by qPCR. As shown in FIG. 2, panel F, the level miR-148a in the serum of AD patients was significantly reduced compared with HAVs, indicating that the downregulation of miR-148a is closely related to AD (mean±SEM, n=5 to 14, **P<0.01).

### Example 5 Aberrant Expression of miR-148a in In Vitro VaD Models

Five mM sodium dithionite (Na₂S₂O₄) was used to damage SH-SYSY cells to establish an oxygen-glucose deprivation (OGD) cell model to simulate the pathological state of VaD. After the Na₂S₂O₄ injury, RNA was extracted, and the expression of miR-148a was detected by qPCR. As shown in FIG. 2, panel G, the expression of miR-148a in Na₂S₂O₄-injured cells was significantly decreased than that of control cells (mean±SEM, n=4, *P<0.05).

### Example 6 Aberrant Expression of miR-148a in VaD Animal Models

SD rats suffering from 2-vessel occlusion (2VO) were used to establish the VaD model, and the expression changes of miR-148a were detected in the cerebral cortex and hippocampus of rats with VaD. As shown in FIG. 2, panel H, the expression of miR-148a in the cortex and hippocampus of rats with 2V0 was significantly downregulated compared with that of rats in the sham group (mean±SEM, n=6, *P<0.05), indicating that the expression of miR-148a is downregulated during VaD pathology.

### Example 7 Effect of miR-148a Upregulation on the Viability of Nerve Cells

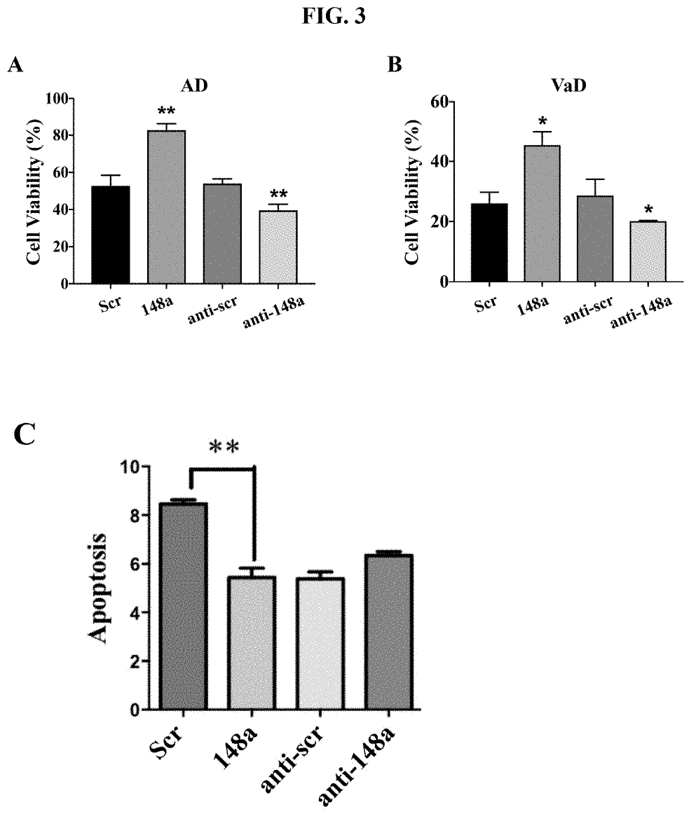

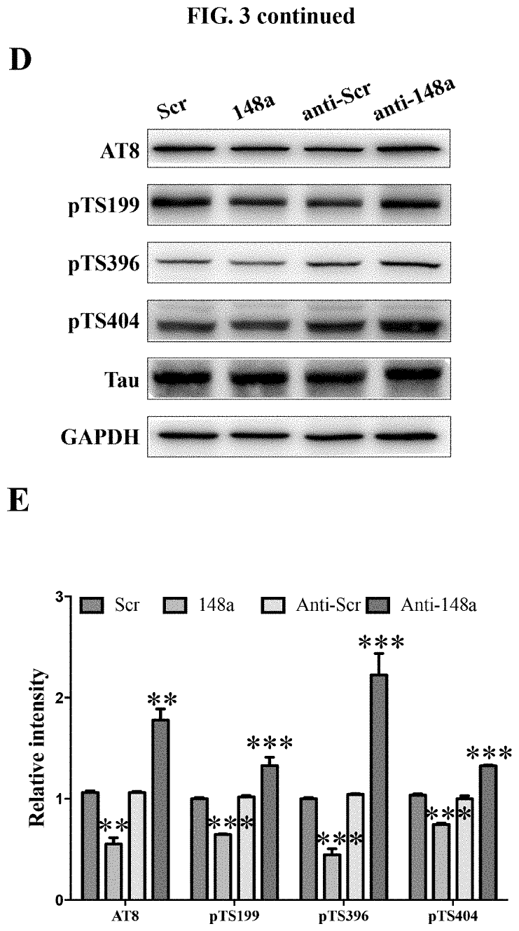

In order to explore the neuroprotective effect of miR-148a in AD and VaD, two cell models were transfected with miR-148a mimics or inhibitor, and the CCK-8 was used to detect cell viability. As shown in FIG. 3, panels A-B, the upregulation of miR-148a significantly increased the cell viability (mean±SEM, n=4, *P<0.05, **P<0.01), and the downregulation of miR-148a significantly reduced the cell viability (mean±SEM, n=4, *P<0.05, **P<0.01), indicating that miR-148a has a neuroprotective effect on AD and VaD cell models.

### Example 8 Effect of miR-148a Upregulation on the Apoptosis of Nerve Cells

The apoptosis rate was detected by flow cytometry to further confirm the role of miR-148a in nerve cells. As shown in FIG. 3, panels C-D, the upregulation of miR-148a significantly inhibited the apoptosis of APPswe cells (mean±SEM, n=4, **P<0.01), while downregulation of miR-148a expression increased the apoptosis rate (mean±SEM, n=4).

### Example 9 miR-148a Inhibits the Hyperphosphorylation of Tau

In order to explore the role of miR-148a in the phosphorylation of Tau, APPswe cells were transfected with miR-148a mimics or inhibitor, and Western blot (WB) was used to detect the phosphorylation level of Tau at various sites. As shown in FIG. 3, panels E-F, the overexpression of miR-148a could significantly inhibit the phosphorylation level of Tau at AT8, Ser199, Ser396 and Ser404 sites; and conversely, inhibiting the expression of miR-148a could significantly increase the phosphorylation level at these sites (mean±SEM, n=6, **P<0.01, ***P<0.001), indicating that miR-148a has an excellent inhibitory effect on Tau phosphorylation.

### Example 10 Directly Binding of miR-148a to p35 (CDK 5 Regulatory Subunit 1)

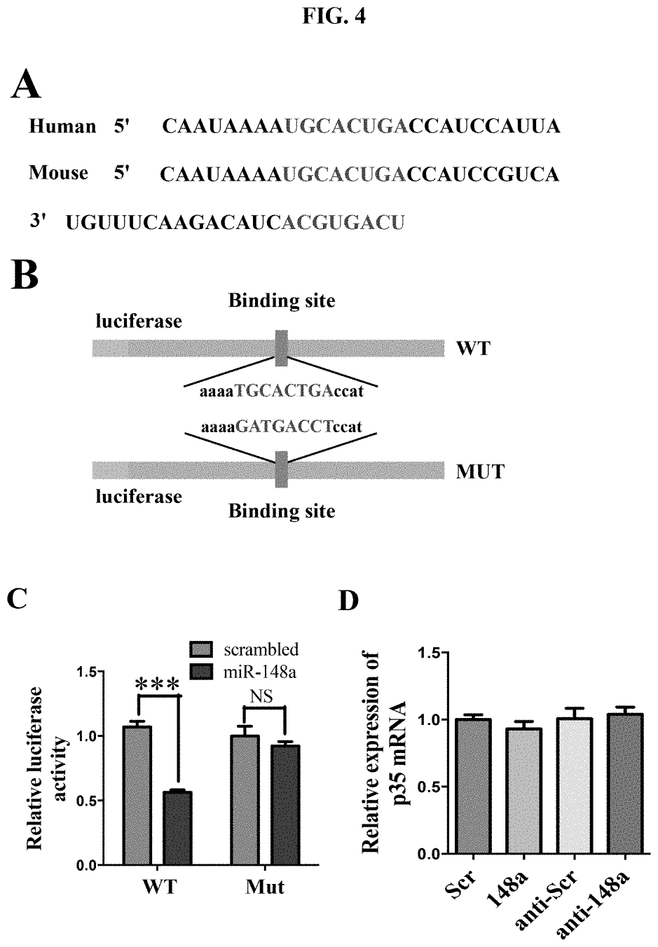

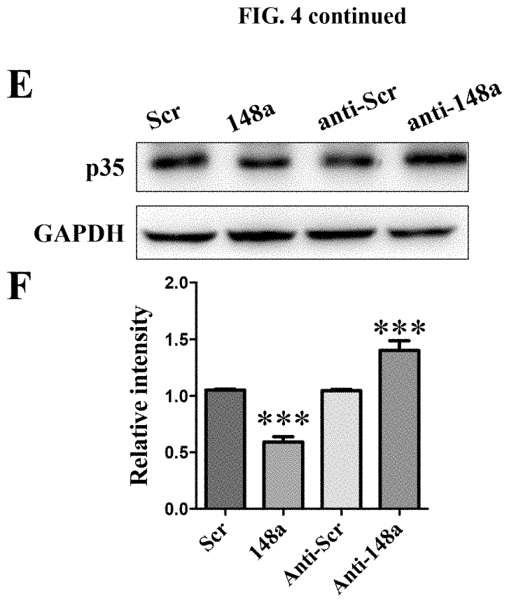

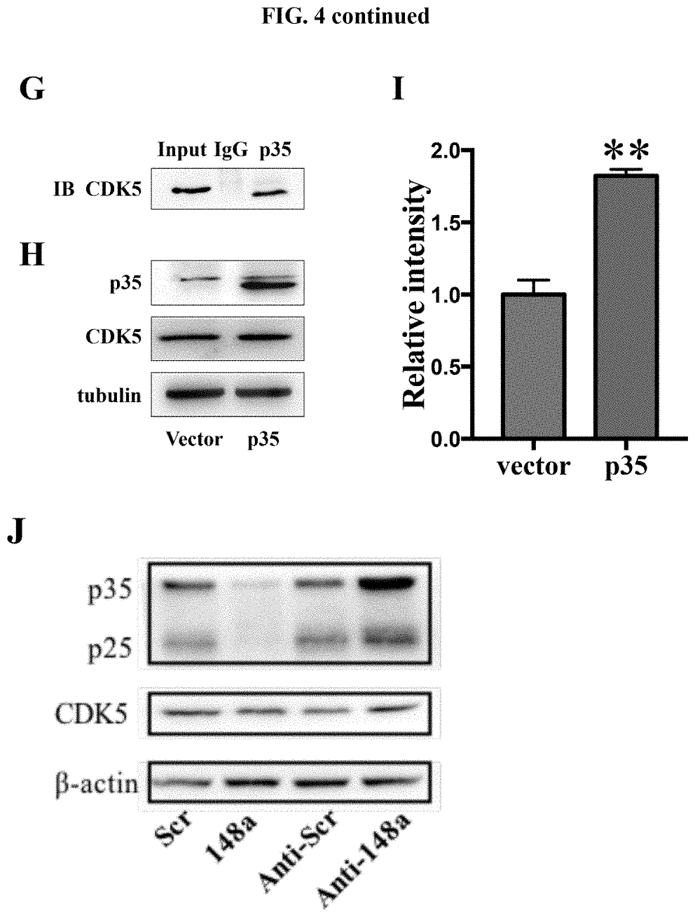

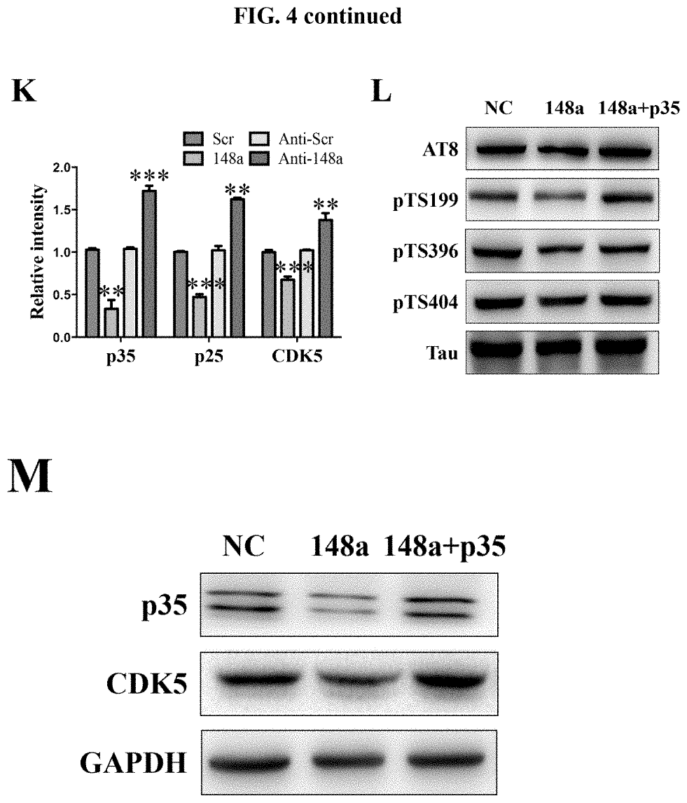

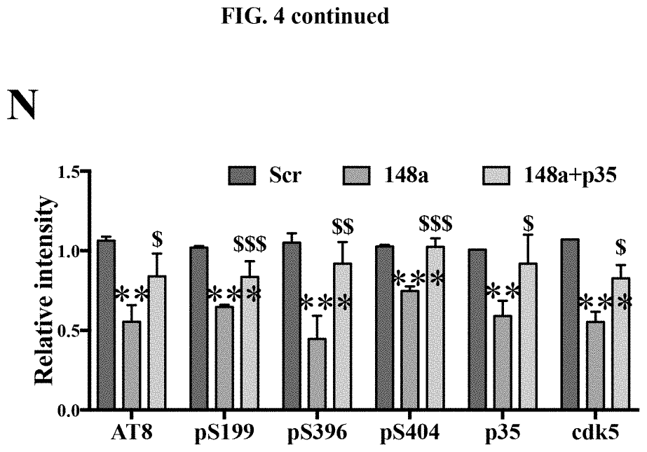

In order to explore the inhibitory mechanism of miR-148a on Tau phosphorylation, bioinformatics software was used to explore its target, and it was found that miR-148a could specifically bind to the 3′UTR of p35 mRNA, and the binding site as shown in FIG. 4, panel A was conservative in humans and mice.

As shown in FIG. 4, panel B, a dual-luciferase reporter gene plasmid was constructed according to the binding site. The WT binding site or a mutant binding site was cloned into a site behind a luciferase fragment and co-transfected with the Renilla plasmid and miR-148a mimics into HEK293 cells. As shown in FIG. 4, panel C, miR-148a significantly reduced the luciferase activity at a WT site but exhibited no effect on the luciferase activity at the mutant site (mean±SEM, n=6, ***P<0.001). This proves that miR-148a can specifically bind to 3′UTR of p35 mRNA.

### Example 11 Regulatory Effect of miR-148a on p35 Expression

Furthermore, qPCR and WB were used to detect the regulation of miR-148a on the expression of p35 mRNA and protein. As shown in FIG. 4, panels D-F, compared with the control group, the upregulation of miR-148a significantly reduced the expression of p35 protein, while downregulation of miR-148a significantly increased the expression of p35 protein (mean±SEM, n=6, ***P<0.001); miR-148a exhibited no effect on the mRNA expression of p35 (mean±SEM, n=6). It shows that miR-148a inhibits the translation process of p35, but does not affect the stability of p35 mRNA.

### Example 12 p35 Regulates the Expression of CDK5 by Directly Binding

CDK5 is a member of the cyclin-dependent kinase family, which does not regulate the cell cycle and is an important kinase for Tau in nerve cells. p35 is a specific activator for CDK5 in the brain. In order to study the effect of p35 on CDK5, a p35 plasmid was overexpressed in SH-SYSY cells, and the expression changes of CDK5 were detected by co-immunoprecipitation (CO-IP) and WB assay. As shown in FIG. 4, panels G-I, p35 indeed interacted with CDK5 (mean±SEM, n=6), and the overexpression of p35 significantly increased the expression of CDK5 in cells (mean±SEM, n=6, **P<0.01). These results indicate that p35 may directly interact with CDK5 to regulate the expression of CDK5, thereby regulating the phosphorylation level of Tau.

### Example 13 Regulation of miR-148a on the Expression of CDK5 and the Phosphorylation of Tau Depends on the Regulation on p35

In order to further study the potential mechanism of miR-148a regulating Tau phosphorylation, the expression of miR-148a was upregulated in cells to detect the expression levels of p35, p25, and CDK5. As shown in FIG. 4, panels J-K, upregulation of miR-148a in vitro significantly reduced the expression levels of p25 and CDK5, while downregulation of miR-148a resulted in significantly higher expression levels of p25 and CDK5 in vitro than the control group (mean±SEM, n=6, **P<0.01, ***P<0.001). When the expression of miR-148a was upregulated, the expression level of p35 decreased the most, the expression level of p25 decreased the medium, and the expression level of CDK5 decreased the least. Similarly, when the expression of miR-148a was inhibited, the expression level of p35 increased the most, the expression level of p25 increased the medium, and the expression level of CDK5 increased the least. It is inferred from above that miR-148a can secondarily affect p25 and CDK5 by directly regulating the expression of p35, thereby regulating the phosphorylation level of Tau in vitro.

In order to verify the above inference, the cells were simultaneously transfected with miR-148a and p35. As shown in FIG. 4, panels L-N, the overexpression of p35 reversed the decrease in the Tau phosphorylation level caused by the upregulation of miR-148a. Similarly, the overexpression of p35 could also reverse the decrease in expression levels of p35, p25 and CDK5 (mean±SEM, n=4, **P<0.01, ***P<0.001, ^{$}P<0.05, ^{$$}P<0.01, ^{$$$}P<0.001). It is concluded that miR-148a can inhibit the CDK5-induced hyperphosphorylation of Tau by targeting p35 to ultimately exert a neuroprotective effect.

### Example 14 Overexpression of miR-148a in the Brain Improves Cognitive Impairment of APP/PS1 Mice

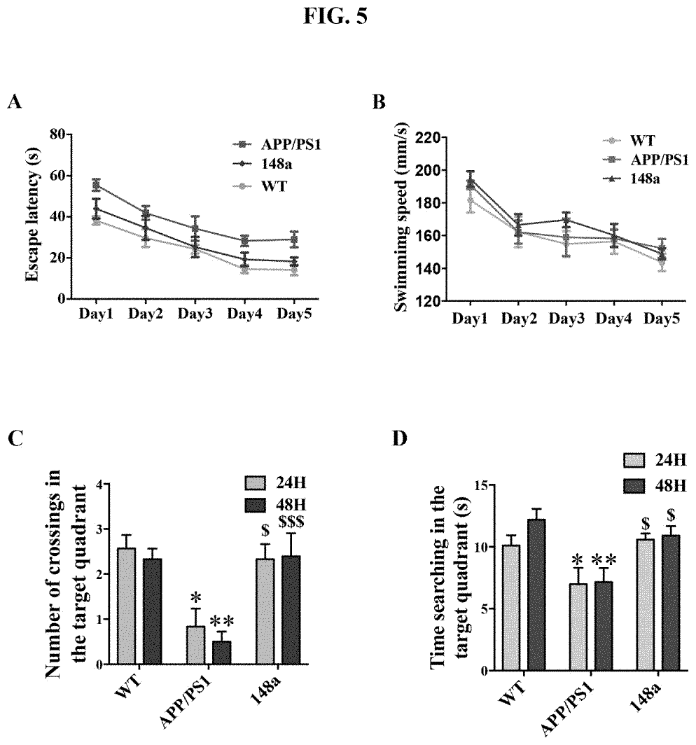

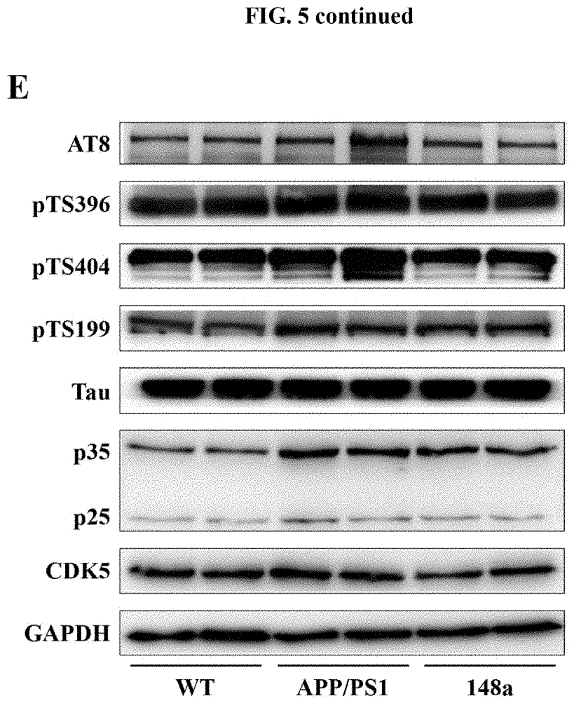

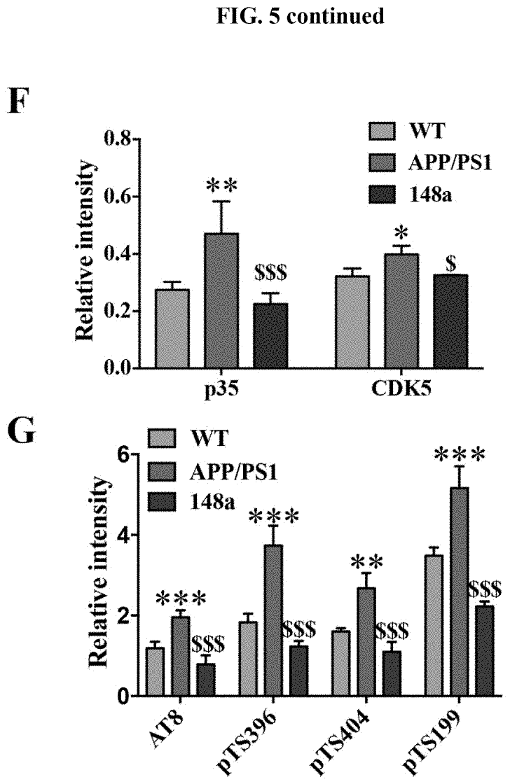

APP/PS1 mice are commonly used AD animal models, which can well simulate the pathology of AD at advanced stage. 6-month-old APP/PS1 mice were selected for test. The brain of APP/PS1 mice was intracerebroventricularly injected with miR-148a adeno-associated virus (AAV) to upregulate the expression of miR-148a in the brain. The Morris water maze experiment was conducted to explore the effect of miR-148a on the cognition of mice. As shown in FIG. 5, panels A-D, the spatial learning and memory impairment in APP/PS1 mice could be alleviated and improved by miR-148a, which was specifically expressed as: in the place navigation test, the five-day escape latency of the APP/PS1 mice was significantly longer than that of the WT control group, while the escape latency of mice in the miR-148a treatment group was shorter than that of APP/PS1 mice, and a significant difference appeared on day 5 (mean±SEM, n=10, *P<0.05); and in the probe trial, the duration within the target quadrant and numbers of crossings through platform location of APP/PS1 mice were significantly reduced compared with the WT control group, while those of miR-148a treatment group were significantly increased compared with the APP/PS1 control mice (mean±SEM, n=10, *P<0.05, **P<0.01, ^{$}P<0.05, ^{$$$} P<0.001). In addition, there was no difference in the swimming speed of mice in the three groups in the five-day place navigation test (mean±SEM, n=10). It can be suggested that miR-148a can improve the spatial learning and memory capacity of AD mice.

### Example 15 miR-148a Relies on p35 to Significantly Reduce the Phosphorylation of Tau in the Brain of APP/PS1 Mice

Based on the discovery of the regulatory relationship between miR-148a and p35, p25 or CDK5 in vitro, the correlation between miR-148a and p35, p25 or CDK5 was further tested in vivo. As shown by results in FIG. 5, panels E-F, compared with WT control mice, the p35 and CDK5 levels in the hippocampus of APP/PS1 mice were significantly increased, while the p35 and CDK5 protein levels in the hippocampus of the miR-148a-treated mice were significantly reduced (mean±SEM, n=5, *P<0.05, **P<0.01, ^{$}P<0.05, ^{$$$}P<0.001), indicating that miR-148a can regulate the expression of p35 and CDK5 in the brain of AD model animals.

Since miR-148a was previously found to affect the pathological change of Tau hyperphosphorylation in AD model cells, the regulation of miR-148a on Tau phosphorylation was further observed in the hippocampus of AD mice. As shown in FIG. 5, panel E&G, the phosphorylation level of Tau in the hippocampus of APP/PS1 mice was significantly higher than that in WT mice, and the upregulation of miR-148a could significantly reduce the phosphorylation levels at AT8, Ser199, Ser396, and Ser404 sites in the hippocampus of APP/PS1 mice (mean±SEM, n=5, **P<0.01, ***P<0.001, ^{$$$}P<0.001). The above results indicate that miR-148a regulates the expression of p35 to inhibit the expression of p25 and CDK5, reduce the hyperphosphorylation level of Tau, and improve the cognitive impairment in AD mice.

### Example 16 Detection of Aberrant Expression of PTEN (Phosphatase and Tensin Homologs Deficient by Chromosome 10) in Brain Tissues of AD Model Animals and WT Animals by mRNA Microarray Technology

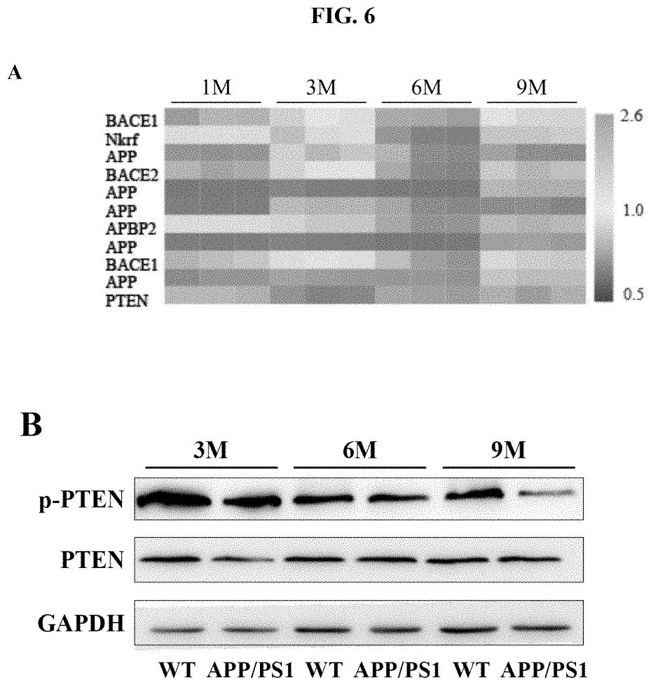

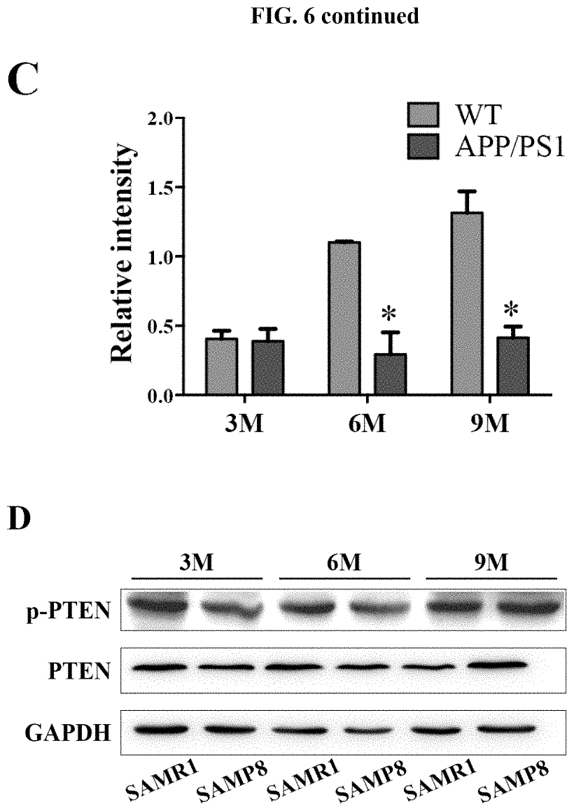

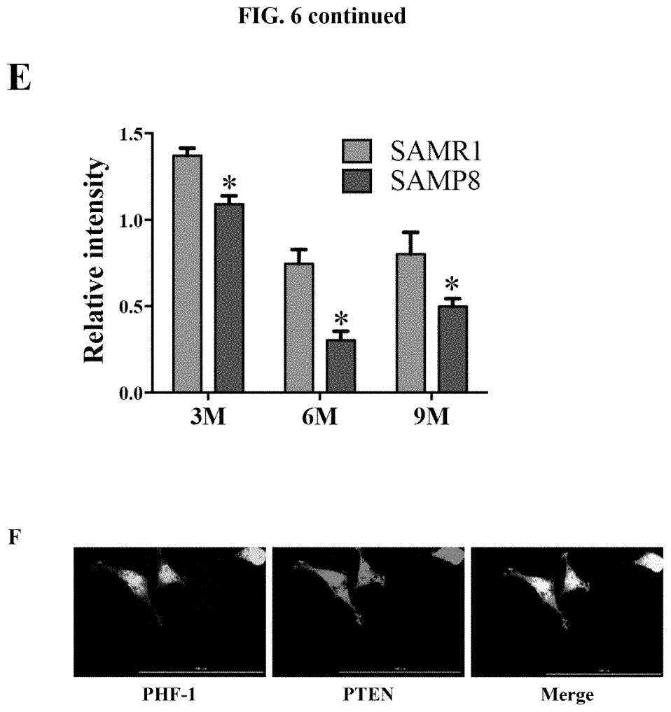

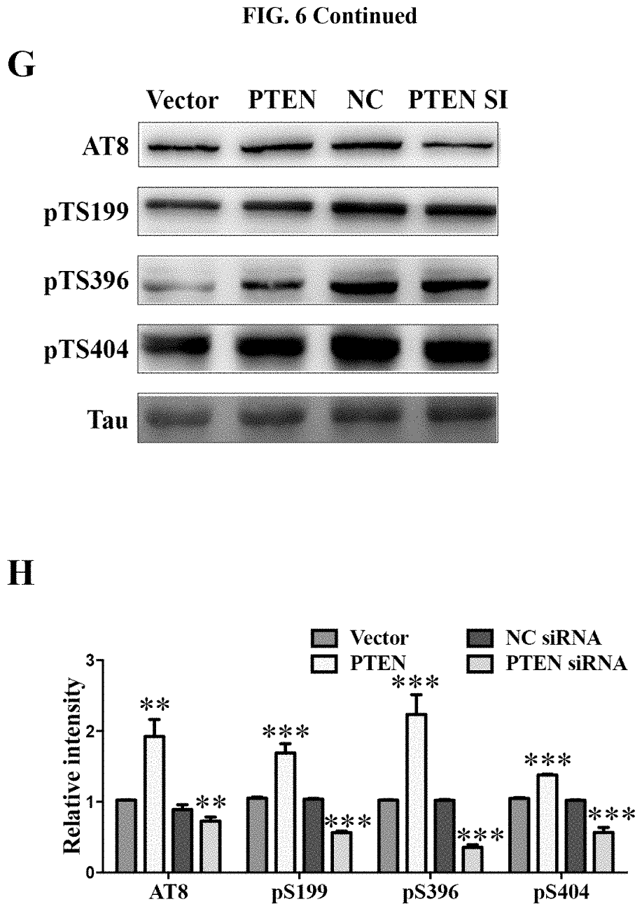

RNA was extracted from brain tissues of 1-month-old, 3-month-old, 6-month-old, and 9-month-old APP/PS1 double-transgenic mice and WT control mice therefore, and the mRNA was fluorescently labeled using the Quick Amp Labeling Kit. PTEN gene qPCR primers involved in this example: forward primer: SEQ ID NO. 6: attggctgctgtcctgctgtt; and reverse primer: SEQ ID NO. 7: ggttaagtcattgctgctgtgtct. Then Agilent Microarray Scanner was used to scan the array, and Agilent Feature Extraction software was used for data acquisition and analysis. The array results in FIG. 6, panel A, show the differential expression of six AD-related genes including PTEN in APP/PS1 mice at different ages. PTEN was significantly downregulated in the brain of 1-month-old, 3-month-old, 6-month-old, and 9-month-old APP/PS1 mice, and the relative upregulation was the largest in the brain of 3-month-old mice (mean±SEM, n=3, fold change>2).

### Example 17 Aberrant Expression of PTEN in AD Model Animals and SAMP8 Strain

In order to further confirm the expression changes of PTEN in the brain tissues of AD animals, the expression of PTEN protein in the brain of 3-month-old, 6-month-old, and 9-month-old APP/PS1 mice and SAMP8 mice was detected. As shown in FIG. 6, panels B-E, although the total amount of PTEN protein in the brain of 3-month-old, 6-month-old, and 9-month-old APP/PS1 mice did not change much, the phosphorylated PTEN protein in the brain of the 6-month-old and 9-month-old APP/PS1 mice was significantly reduced (mean±SEM, n=5, *P<0.05), so the proportion of non-phosphorylated PTEN protein increased significantly, indicating that the expression of PTEN of the active form increased. Similarly, in the brain of SAMP8 mice, the expression of phosphorylated PTEN protein in the brain of 3-month-old, 6-month-old, and 9-month-old mice significantly decreased (mean±SEM, n=5, *P<0.05), so the expression of PTEN of the active form significantly increased. Therefore, it is inferred that the activity of PTEN in AD is significantly increased, which may play an important role in AD pathology.

### Example 18 Effect of Changes in the PTEN Expression Level on the Phosphorylation of Tau

In order to explore the relationship between PTEN and Tau phosphorylation, the immunofluorescence technology was used to analyze the localization of PTEN and phosphorylated Tau in cells. As shown by results in FIG. 6, panel F, the phosphorylated Tau PHF-1 was mainly present in the cytoplasm and was rarely distributed in the synapse, while PTEN was distributed in both the cytoplasm and synapse; and the expression of the two had overlapped positions, indicating that PTEN is closely related to the content and distribution of phosphorylated Tau.

In order to further explore the effect of PTEN on the Tau phosphorylation, the PTEN was transfected into APPswe cells. As shown by results in FIG. 6, panels G-H, compared with the control group, the upregulation of PTEN could significantly increase the phosphorylation levels at AT8, Ser199, Ser396 and Ser404 sites in vitro (mean±SEM, n=6, **P<0.01, ***P<0.001); and similarly, after PTEN was downregulated in cells, the phosphorylation levels at these sites were significantly reduced (mean±SEM, n=6, **P<0.01, ***P<0.001). It is inferred from above that PTEN can regulate the Tau phosphorylation level in vitro, thereby affecting the pathological process of AD.

### Example 19 PTEN Signaling Pathway Downregulates the Expression of miR-148a

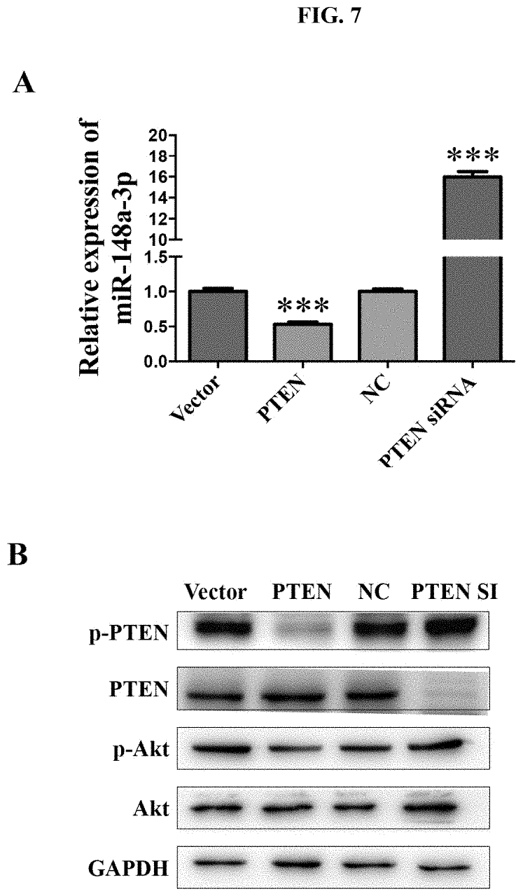

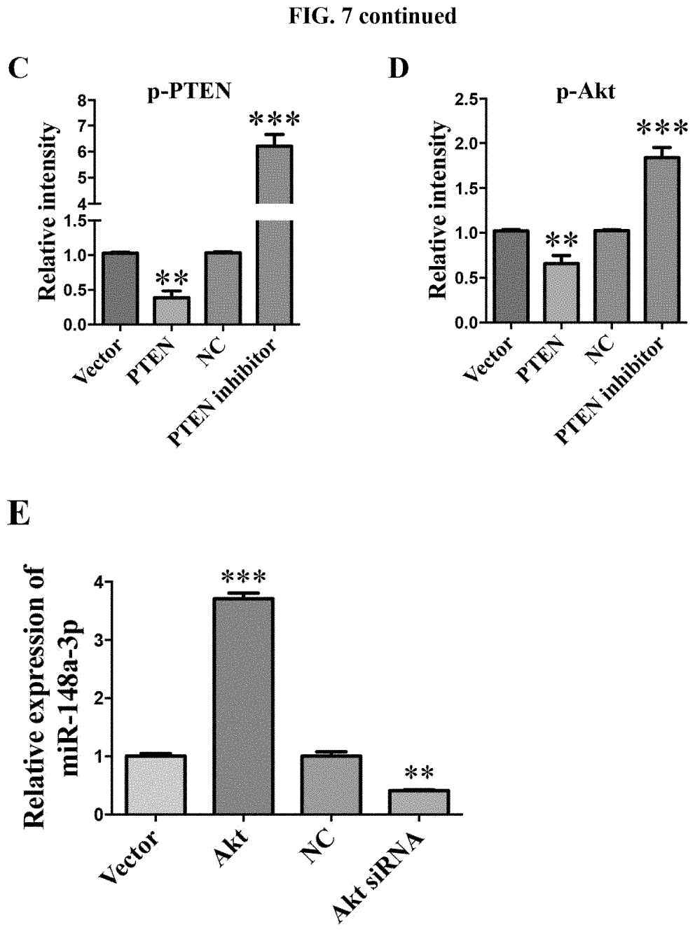

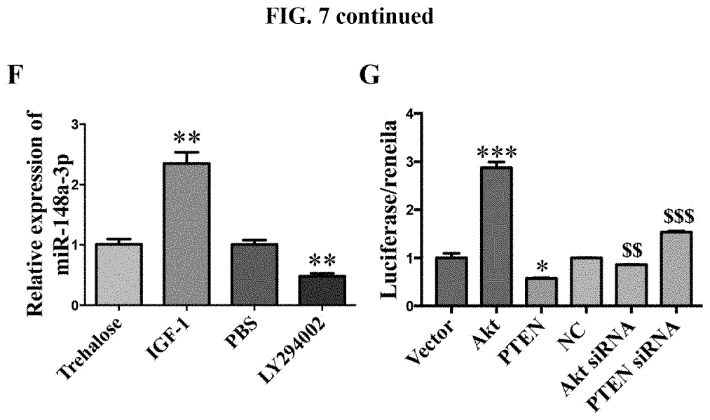

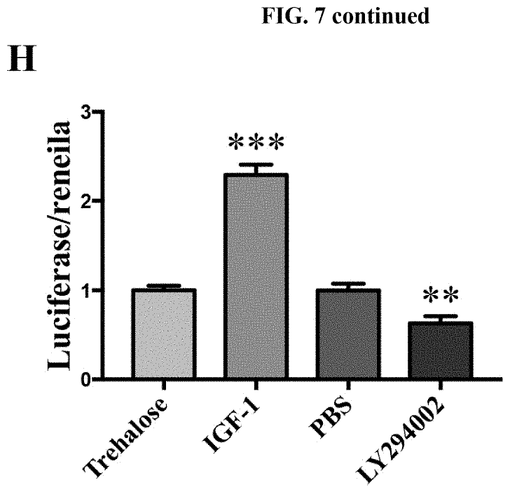

In order to explore the relationship between PTEN and miR-148a, the plasmid or siRNA was transfected into APPswe cells to overexpress or inhibit the expression of PTEN, and the expression changes of miR-148a were detected by the qPCR. As shown in FIG. 7, panel A, when PTEN was overexpressed, the expression of miR-148a in cells was significantly reduced; and when the expression of the PTEN was inhibited, the expression of miR-148a was significantly increased (mean±SEM, n=4, ***P<0.001). This suggests that PTEN may downregulate the expression of miR-148a.

### Example 20 PTEN Signaling Pathway Downregulates the Expression of Akt (Protein Kinase B) Signaling Pathway

As an inhibitor of Akt signaling pathway, PTEN downregulates the downstream pathway of Akt. As shown in FIG. 7, panels B-D, transfecting the PTEN plasmid into cells could significantly reduce the ratio of p-PTEN/PTEN and p-Akt/Akt (mean±SEM, n=6, **P<0.01). This proves that PTEN plasmid transfection can increase the activity of PTEN and reduce the activity of Akt. Conversely, PTEN siRNA could significantly increase the specific ratio of p-PTEN/PTEN and p-Akt/Akt, proving that PTEN siRNA can reduce the activity of PTEN and increase the activity of Akt (mean±SEM, n=6, ***P<0.001).

### Example 21 Akt Signaling Pathway Upregulates the Expression of miR-148a

In order to explore the effect of Akt signaling pathway on the expression of miR-148a, the cells were transfected with the Akt plasmid and Akt siRNA to change the expression of Akt. As shown in E of FIG. 7, panel E, when the Akt expression was upregulated, the expression of miR-148a was significantly increased, and when the Akt expression was decreased, the expression of miR-148a was significantly decreased (mean±SEM, n=4, **P<0.01, ***P<0.001). Similarly, IGF-1 (an activator of Akt signaling pathway) and LY294002 (an inhibitor of Akt signaling pathway) were added to cells to increase or decrease the Akt expression. As shown in FIG. 7, panel F, the expression level of miR-148a in IGF-1-treated cells increased by 2.5 folds compared with that in trehalose-treated cells, while the expression level of miR-148a in LY294002-treated cells was reduced by one fold compared with that in the control group (mean±SEM, n=4, **P<0.01). It can be seen that the activation of the Akt signaling pathway can promote the expression of miR148a, that is, the Akt signaling pathway presents a positive regulatory relationship with the expression level of miR-148a.

### Example 22 PTEN/Akt Signaling Pathway Regulates the Transcription of miR-148a

The above experiments suggest that the PTEN/Akt signaling pathway may affect the expression of miR-148a by regulating the transcription process of miR-148a. Therefore, a miR-148a promoter region luciferase plasmid was constructed. The increased luminescence value indicates that the miR-148a transcription is promoted, and the decreased luminescence value indicates that the miR-148a transcription is inhibited. As shown in FIG. 7, panels G-H, compared with the control group, the upregulation of PTEN expression significantly inhibited the transcription level of miR-148a, while the downregulation of PTEN expression significantly increased the transcription level of miR-148a (mean±SEM, n=4, *P<0.05, ^{$s$}P<0.001). The overexpression of Akt increased the luminescence value by 2.8 folds, and adding IGF-1 to activate the Akt signaling pathway could also increase the luminescence value by 2.2 folds; and conversely, inhibiting the expression of Akt significantly reduced the luminescence value, and adding LY294002 to inhibit the Akt signaling pathway also significantly downregulated the luminescence value (mean±SEM, n=4, **P<0.01, ***P<0.001, ^{$$}P<0.01). The above results indicate that the PTEN/Akt signaling pathway regulates the transcription of miR-148a.

### Example 23 CREB Specifically Binds to the miR-148a Promoter Region

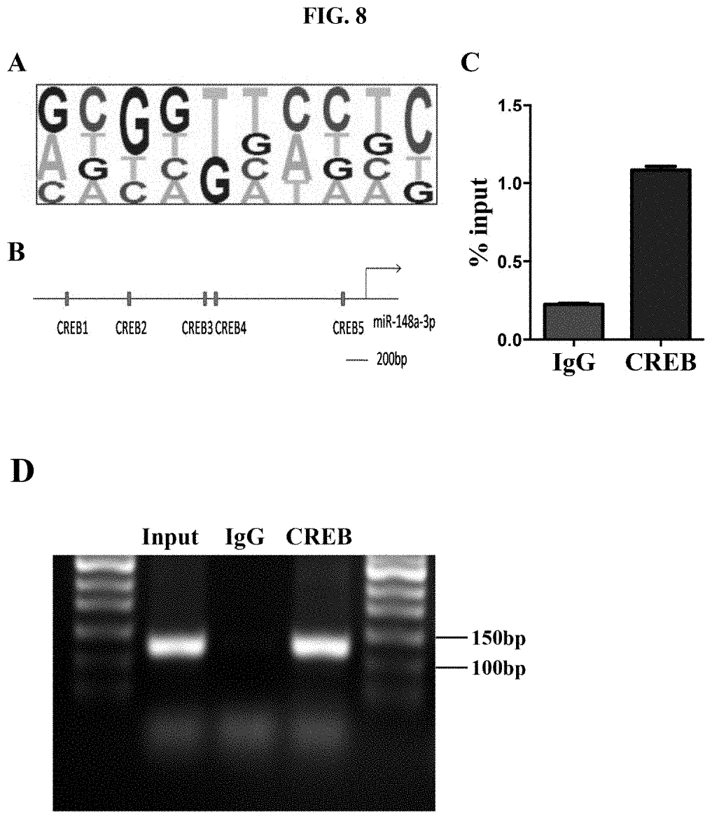

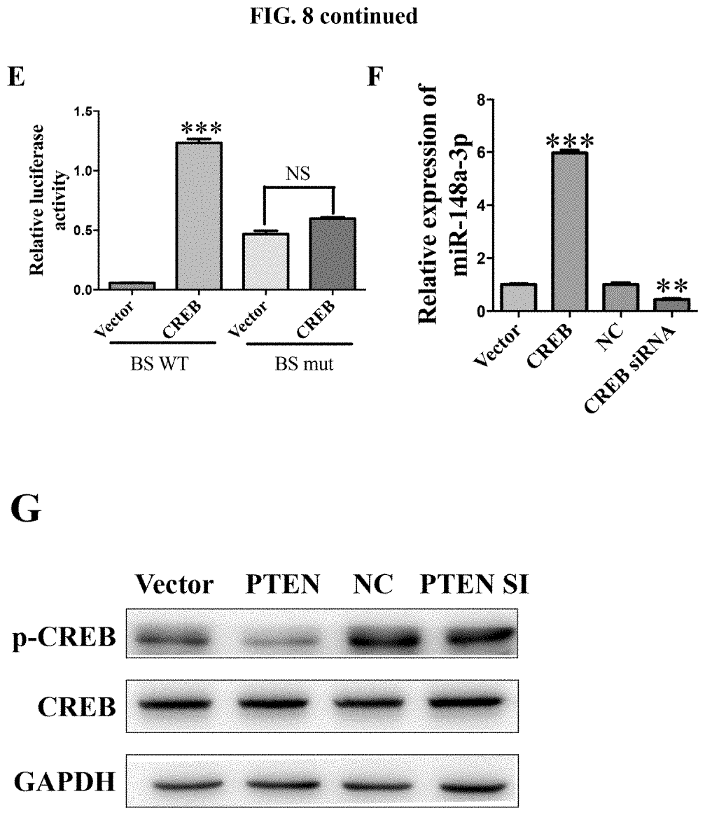

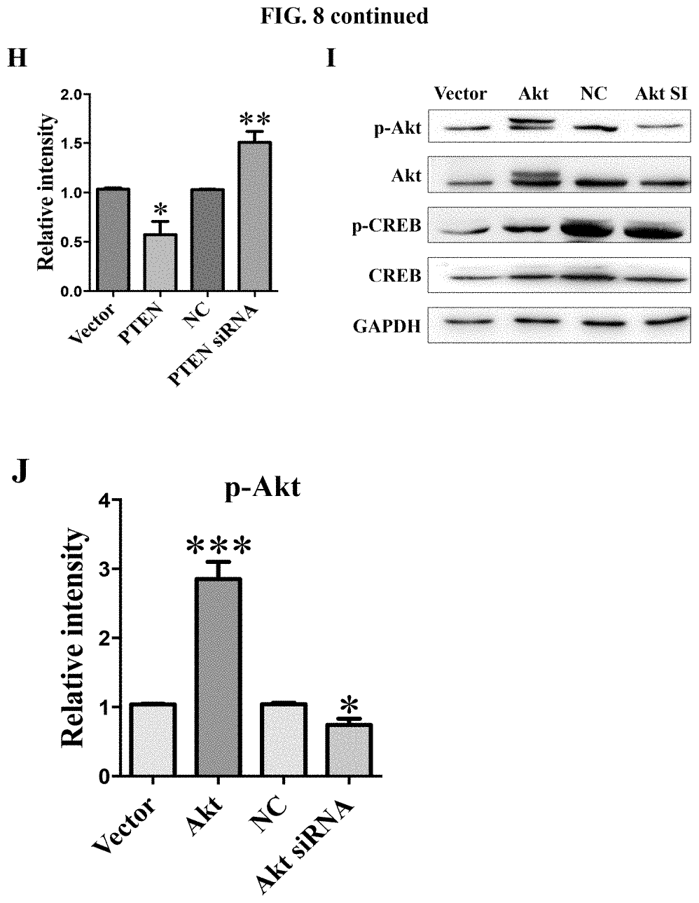

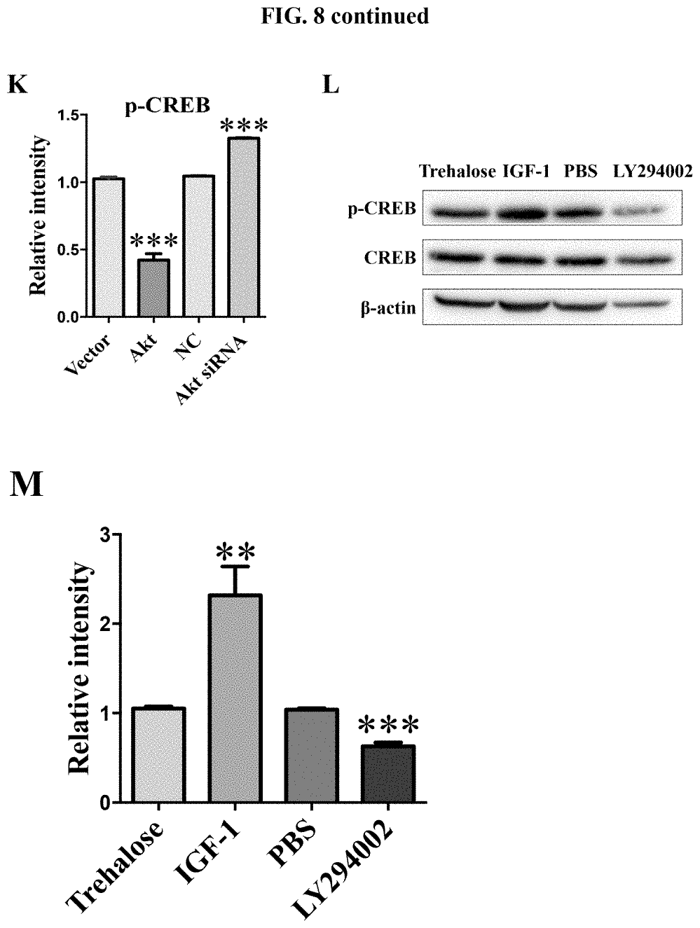

Since the PTEN/Akt signaling pathway can regulate the transcription of miR-148a, the promoter region of miR-148a was analyzed. Promoter Scan software found that the promoter region of miR-148a may bind to CREB. FIG. 8, panel A shows the possible binding sites of CREB to the promoter region of miR-148a. Primers were designed for the predicted five sites (FIG. 8, panel B) and the chromatin immunoprecipitation (ChIP) test was conducted to find possible sites for directly binding. As shown in FIG. 8, panels C-D, in the ChIP test, DNA immunoprecipitated by the CREB antibody was subjected to qPCR, and results confirmed that the quantity of DNA immunoprecipitated by the CREB antibody was more than five times that of DNA immunoprecipitated by the IgG antibody; and the agarose gel electrophoresis test confirmed that the quantity of DNA immunoprecipitated by the CREB antibody was significantly higher than that of DNA immunoprecipitated by the IgG antibody, and the length of the fragment was consistent with the primer amplification length (mean±SEM, n=3). It is inferred that CREB may bind to the miR-148a promoter region at the 1217th base behind the transcription initiation site.

### Example 24 CREB Upregulates the Transcription and Expression of miR-148a

In order to further verify whether CREB can regulate the transcription of miR-148a, a dual-luciferase reporter gene was designed according to the binding site. As shown in FIG. 8, panel E, CREB could increase the luminescence value of the WT luciferase plasmid, but exhibited no effect on the luminescence value of the mutant plasmid (mean±SEM, n=6, ***P<0.001). CREB overexpression plasmid and CREB siRNA were used to change the expression of CREB in cells, and the expression of miR-148a was detected by the qPCR. As shown in FIG. 8, panel F, upregulating the expression level of CREB could increase the expression of miR-148a by 5 folds; and conversely, when the expression of CREB was inhibited, the expression level of miR-148a was significantly reduced (mean±SEM, n=6, **P<0.01, ***P<0.001). The above results suggest that CREB can not only directly bind to the promoter region of miR-148a, but also regulate the transcription and expression levels of miR-148a.

### Example 25 PTEN/Akt Signaling Pathway Regulates the Expression of CREB

Since CREB can directly bind to the promoter region of miR-148a, it can regulate the transcription of miR-148a. Combining the above experimental results, the regulatory effect of PTEN/Akt signaling pathway on CREB was further studied. The cells were transfected with a PTEN expression plasmid and PTEN siRNA, and the WB was used to detect changes in the activity of CREB. As shown in FIG. 8, panels G-H, when the expression of PTEN was upregulated, the ratio of p-CREB/CREB was halved; and when the expression of PTEN was decreased, the ratio of p-CREB/CREB was significantly increased (mean±SEM, n=6, *P<0.05, **P<0.01).

The expression of Akt in cells was changed to detect expression changes of CREB. As shown in FIG. 8, panels I-M, when the expression of Akt increased or when Akt was activated by IGF-1, the ratio of p-CREB/CREB increased significantly; and when the expression of Akt was inhibited or when LY294002 was used to inhibit the Akt signaling pathway, the ratio of pCREB/CREB was significantly reduced (mean±SEM, n=6, **P<0.01, ***P<0.001). It is inferred that the PTEN/Akt signaling pathway affects the transcription of miR-148a by regulating the expression of CREB, thereby affecting the phosphorylation level of Tau.

### Example 26 Inhibiting the Expression of PTEN in the Brain of APP/PS1 Mice can Improve the Learning and Memory Capacity of APP/PS1 Mice

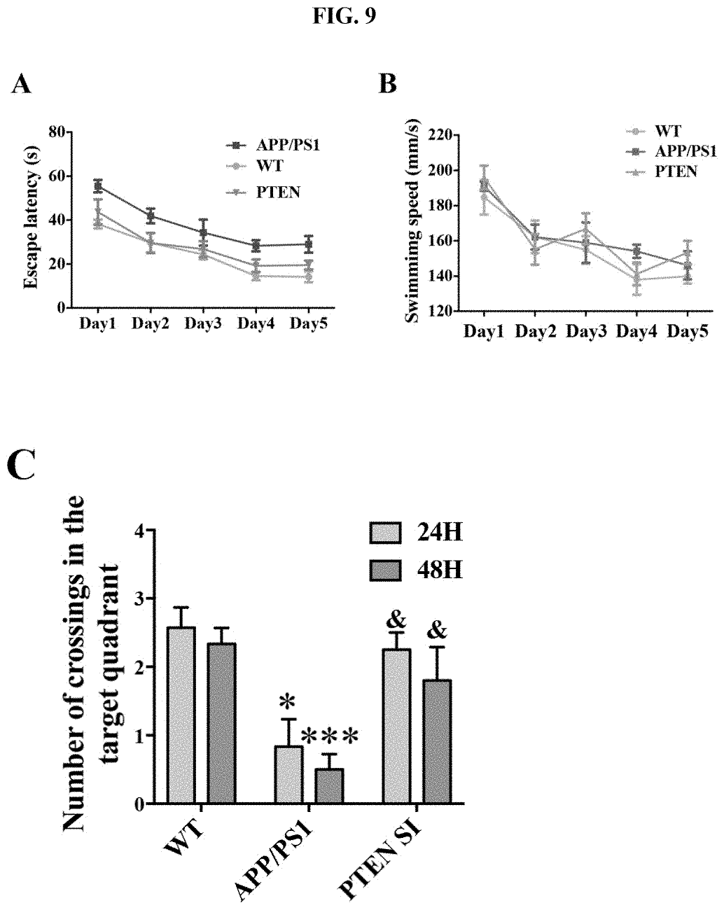

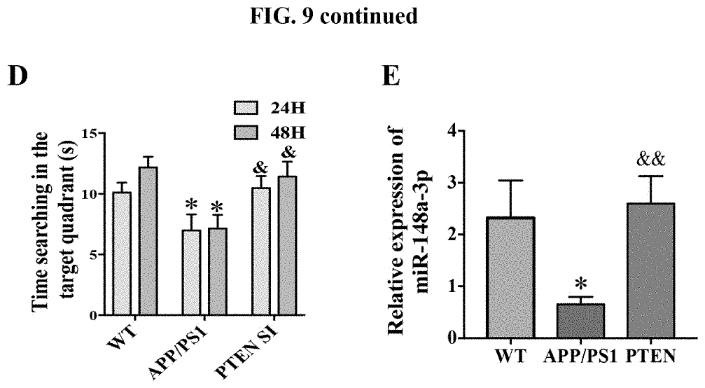

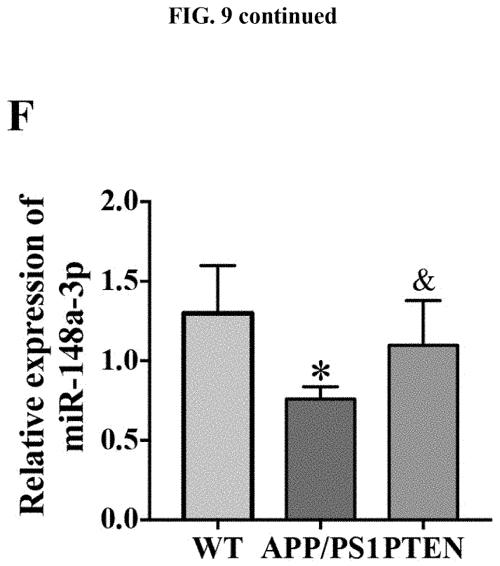

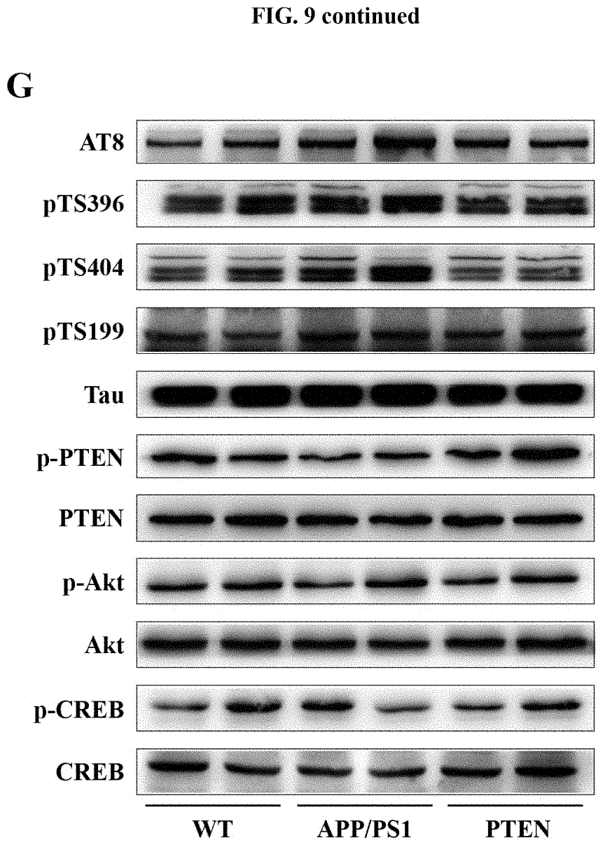

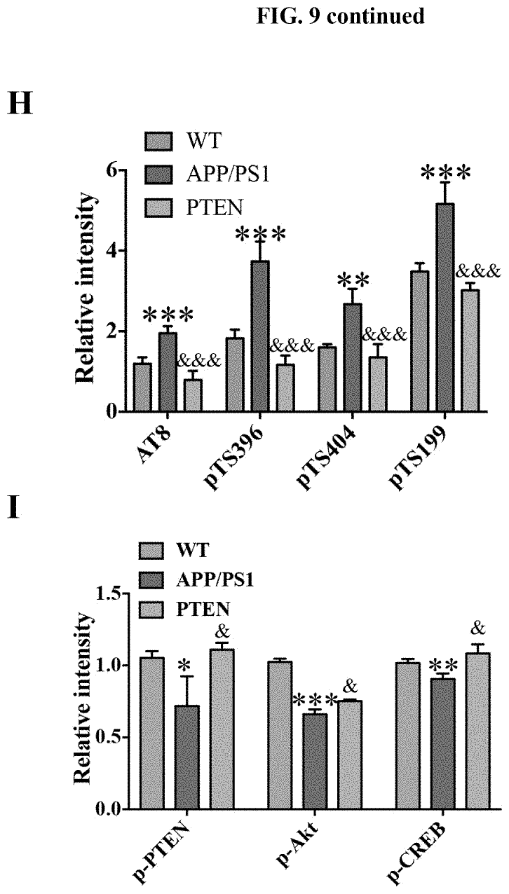

In order to further study the role of PTEN in AD, the brains of APP/PS1 mice were intracerebroventricularly injected with PTEN siRNA AAV and control AAV, separately. Thirty days after the injection, the Morris water maze experiment was conducted to determine the effect of PTEN on the learning and memory capacity of AD mice. As shown in FIG. 9, panels A-D, in the five-day place navigation test, escape latency of APP/PS1 mice far exceeded that of WT mice, proving that APP/PS1 mice suffering from learning and memory impairment; but in APP/PS1 mice injected with PTEN siRNA, the memory impairment was significantly improved, and the escape latency of mice in this group was significantly shortened (mean±SEM, n=10, *P<0.05). In addition, there was no difference in swimming speed among all groups, which avoided the difference in escape latency caused by physical factors of mice (mean±SEM, n=10). In the probe trial, the duration within the target quadrant and numbers of crossings through platform location of APP/PS1 control mice were significantly lower than that of the WT control group, while the those were significantly increased in the APP/PS1 mice treated with PTEN siRNA. It is proved that the inhibition of PTEN can improve the spatial learning and memory capacity of AD mice (mean±SEM, n=10, *P<0.05, ***P<0.001, ^{&}<0.05).

### Example 27 Inhibiting the Expression of PTEN in the Brain of APP/PS1 Mice can Increase the Expression of miR-148a

The qPCR was used to detect the expression level of miR-148a in the brain of mice. As shown in FIG. 9, panels E-F, the expression levels of miR-148a in both cortex and hippocampus of APP/PS1 mice were significantly reduced; but after PTEN siRNA treatment, the expression of miR-148a was increased significantly (mean±SEM, n=5, *P<0.05, ^{&}<0.05, ^{&&}P<0.01), suggesting that inhibiting the expression of PTEN in the brain of AD mice can increase the expression level of miR-148a.

### Example 28 Inhibiting the Expression of PTEN in APP/PS1 Mice Brain can Reduce the Phosphorylation Level of Tau

The WB method was used to detect the phosphorylation levels of Tau in the hippocampus of mice in the above three groups. As shown FIG. 9, panels G-H, the phosphorylation levels at AT8, Ser396, Ser404 and Ser199 sites in the hippocampus of APP/PS1 mice were significantly increased, while the phosphorylation levels at the above sites in the hippocampus of PTEN siRNA-treated mice were decreased significantly (mean±SEM, n=5, **P<0.01, ***P<0.001, ^{&&&}P<0.001). It is proved that inhibiting the expression of PTEN in the brain of AD mice can ameliorate the hyperphosphorylation of Tau.

### Example 29 Inhibiting the Expression of PTEN in APP/PS1 Mice Brain can Activate the Akt/CREB Signaling Pathway

Similarly, the WB method was used to detect expression changes of the Akt/CREB signaling pathway in the hippocampus of mice. As shown in FIG. 9, panels G-I, in the brain of APP/PS1 mice, active form PTEN was significantly increased, and the Akt/CREB signaling pathway was significantly inhibited; and in the hippocampus of mice in the PTEN siRNA treatment group, the expression of p-Akt and p-CREB was increased significantly, and the Akt/CREB signaling pathway was activated (mean±SEM, n=5, *P<0.05, **P<0.01, ***P<0.001, ^{&}P<0.05). It can be seen that inhibiting the expression of PTEN in the brain can activate the Akt/CREB signaling pathway, promote cell survival, and improve learning and memory.

The test results of Examples 1 to 29 of the present disclosure show that the expression of the miRNA 148 cluster is significantly reduced during pathological processes of cognitive impairment-associated diseases AD and VaD, and exogenously increasing the expression of miRNA 148a can result in a neuroprotective effect.

In particular, in a pathological process of AD, upregulating the PTEN expression can inhibit the phosphorylation of Akt, which in turn suppresses the phosphorylation of CREB, reduces the transcription and expression of miR-148a, increases the expression of p35, p25, and CDK5, and promotes the phosphorylation of Tau, thus causing cognitive impairment; and inhibiting the expression of PTEN can activate the phosphorylation of Akt/CREB, promote the transcription of miR-148a, and suppress the expression of p35, p25 and CDK5, thereby inhibiting the phosphorylation of Tau and improving cognitive dysfunction. Therefore, the miRNA 148 cluster is expected to become a novel target for the diagnosis and treatment of cognitive impairment-associated diseases.

Although the present disclosure has been described in detail above with general descriptions and specific examples, it will be apparent to those skilled in the art that some modifications or improvements can be made on the basis of the present disclosure. Therefore, all these modifications or improvements made without departing from the spirit of the present disclosure fall within the scope of the present disclosure.

### REFERENCES

[1] Ittner A., Ittner L. M. Dendritic Tau in Alzheimer's Disease. Neuron 2018, 99(1):13-27. doi: 10.1016/j.neuron.2018.06.003.
[2] Tan L., Chen X., Wang W., et al. Pharmacological inhibition of PTEN attenuates cognitive deficits caused by neonatal repeated exposures to isoflurane via inhibition of NR2B-mediated tau phosphorylation in rats. Neuropharmacology 2017, 114:135-145. doi: 10.1016/j.neuropharm. 2016.11.008.
[3] Saton J. I., Kino Y., Niida S. MicroRNA-Seq Data Analysis Pipeline to Identify Blood Biomarkers for Alzheimer's Disease from Public Data. Biomark Insights 2015,10. doi: 10.4137/BMI.S25132. eCollection 2015.
[4] Guo S. L., Ye H., Teng Y., et al. Akt-p53-miR-365-cyclin D1/cdc25A axis contributes to gastric tumorigenesis induced by PTEN deficiency. Nat Commun 2013, 4:2544. doi: 10.1038/ncomms3544.
[5] Ryder J., Su Y., Ni B. Akt/GSK3β serine/threonine kinases: evidence for a signalling pathway mediated by familial Alzheimer's disease mutations. Cell Signal 2004, 16(2):187-200. doi: 10.1016/j.cellsig.2003.07.004.
[6] Gupta A., Dey C. S. PTEN, a widely known negative regulator of insulin/PI3K signaling, positively regulates neuronal insulin resistance. Mol Biol Cell 2012, 23(19):3882-98. doi: 10.1091/mbc.E12-05-0337.
[7] Scott Bitner R. Cyclic AMP response element-binding protein (CREB) phosphorylation: a mechanistic marker in the development of memory enhancing Alzheimer's disease therapeutics. Biochem Pharmacol 2012, 83(6):705-14. doi: 10.1016/j.bcp.2011.11.009.
[8] Hopkins B. D., Hodakoski C., Barrows D., et al. PTEN function: the long and the short of it. Trends Biochem Sci 2014; 39(4): 183-90. doi: 10.1016/j.tibs.2014.02.006.
[9] Zhao J., Qu Y., Wu J., et al. PTEN inhibition prevents rat cortical neuron injury after hypoxia-ischemia. Neuroscience 2013, 238:242-51. doi: 10.1016/j.neuroscience.2013.02.046.
[10] Lee M. S., Kwon Y. T., Li M., et al. Neurotoxicity induces cleavage of p35 to p25 by calpain. Nature 2000, 405:360-364. doi: 10.1038/35012636.
[11] Fisher A., Sananbenesi F., Schrik C., et al. Cyclin-Dependent Kinase 5 Is Required for Associative Learning. J Neurosci 2002, 22:3700-3707. doi: 10.1523/JNEUROSCI.22-09-03700. 2002.
[12] Monaco E. A. Recent evidence regarding a role for Cdk5 dysregulation in Alzheimer's disease. Curr Alzheimer Res 2004:1567-2050. doi: 10.2174/1567205043480519.
[13] Kimura T., Ono T., Takamatsu J., et al. Sequential changes of tau-site-specific phosphorylation during development of paired helical filaments. Dementia 1996, 7(4):177-181. doi: 10.1159/000106875.
[14] Song G., Ouyang G., Bao S. The activation of Akt/PKB signaling pathway and cell survival. J Cell Mol Med 2005, 9(1):59-71. doi: 10.1111/j.1582-4934.2005.tb00337.x.
[15] Deisseroth K., Heist E. K., Tsien R. W. Translocation of calmodulin to the nucleus supports CREB phosphorylation in hippocampal neurons. Nature 1998,392(6672):198-202. doi: 10.1038/32448.

What is claimed is:

1. A product with an active ingredient of a miRNA 148 cluster or an expression promoter thereof, wherein, use of the product comprises the following (a), (b), (c) and/or (d):
(a) inhibiting Tau phosphorylation;
(b) alleviating neurodegeneration and providing a neuroprotective effect;
(c) diagnosing and/or treating cognitive impairment-associated diseases; and
(d) reducing the expression of p35, p25, and CDK5.

2. The product according to claim 1, wherein the miRNA 148 cluster or the expression promoter thereof is used in the following (a), (b), (c), and/or (d):
(a) preparation of a substance that can inhibit phosphorylation of Tau;
(b) preparation of a substance that can alleviate neurodegeneration and has a neuroprotective effect;
(c) preparation of a substance for diagnosing and/or treating cognitive impairment-associated diseases; and
(d) preparation of a substance for reducing the expression of p35, p25, and cyclin-dependent kinase 5 (CDK5).

3. The product according to claim 1, wherein,
the miRNA 148 cluster is selected from hsa-miR-148a, with a nucleotide sequence shown in SEQ ID NO. 1; and
the miRNA148 cluster is selected from hsa-miR-148a-3p, with a nucleotide sequence shown in SEQ ID NO. 2.

4. The product according to claim 1, wherein,
the product for diagnosing cognitive impairment-associated diseases is a detection kit;
the detection kit comprises primers of the miRNA148 cluster; and the assay kit is used to diagnose cognitive impairment-associated diseases, predict the risk of developing cognitive impairment-associated diseases, or predict the outcome of cognitive impairment-associated diseases in patients suffering from or at risk of developing cognitive impairment-associated diseases.

5. The product according to claim 4, wherein,
the primer is used to determine an expression level of the miRNA 148 cluster in a sample.

6. The product according to claim 1, wherein,
the expression level of the miRNA 148 cluster is based on an expression level of the miRNA 148 cluster in a patient and a reference expression level of the miRNA 148 cluster in a healthy subject; and
if the expression level of the miRNA 148 cluster is significantly lower than the reference expression level of the miRNA 148 cluster in a healthy subject, it indicates that the patient has or is at risk of developing a cognitive impairment-associated disease.

7. The product according to claim 1, wherein,
the expression level of the miRNA 148 cluster is determined by a sequencing-based method, an array-based method, or a PCR-based method.

8. The product according to claim 1, wherein,
the expression promoter of the miRNA 148 cluster is at least one of a reagent, a medicament, a preparation, and a gene sequence that promotes the expression or activation of Akt, a reagent, a medicament, a preparation, and a gene sequence that promotes the expression or activation of cAMP-response element binding protein (CREB), and a reagent, a medicament, a preparation, and a gene sequence that inhibits the expression or activation of PTEN;
the Akt and CREB up-regulate the expression of the miRNA 148 cluster; and the PTEN down-regulates the expression of the miRNA 148 cluster.

9. Use of an agonist for a miRNA 148 cluster in the preparation of a medicament for treating cognitive impairment-associated diseases.

10. Use of a long non-coding RNA (lncRNA) interactive with a miRNA 148 cluster in the preparation of a medicament for treating cognitive impairment-associated diseases.


SEQUENCE LISTING (7 sequences)

SEQ ID NO: 1 - RNA, 68 nt, Artificial Sequence
  Nucleotide sequence of hsa-miR-148a
gaggcaaaguucugagacacuccgacucugaguaugauagaagucagugcacuacagaacuuugucuc

SEQ ID NO: 2 - RNA, 22 nt, Artificial Sequence
  Nucleotide sequence of hsa-miR-148a-3p
ucagugcacuacagaacuuugu

SEQ ID NO: 3 - DNA, 50 bp, Artificial Sequence
  Reverse transcription primer
gtcgtatccagtgcagggtccgaggtattcgcactggatacgacacaaag

SEQ ID NO: 4 - DNA, 21 bp, Artificial Sequence
  Forward primer for qPCR
gcgcgtcagtgcactacagaa

SEQ ID NO: 5 - DNA, 20 bp, Artificial Sequence
  Reverse primer for qPCR
agtgcagggtccgaggtatt

SEQ ID NO: 6 - DNA, 21 bp, Artificial Sequence
  Forward primer for qPCR of PTEN gene
attggctgctgtcctgctgtt

SEQ ID NO: 7 - DNA, 24 bp, Artificial Sequence
  Reverse primer for qPCR of PTEN gene
ggttaagtcattgctgctgtgtct